(19) United States
(12) Patent Application Publication
(10) Pub. No.: US 2020/0008668 A1
(43) Pub. Date: Jan. 9, 2020

(54) BIOMEDICAL INFORMATION PROCESSING SYSTEM USING AN APPLICATION PROGRAM COLLABORATED WITH AN OCULAR TEMPERATURE MEASUREMENT TECHNIQUE AND METHOD FOR THE SAME

(71) Applicant: UBIQUITY BIOMEDICAL CORPORATION, TAIPEI CITY (TW)

(72) Inventors: HORNG-JI LAI, TAIPEI CITY (TW); TE-LUN MA, TAIPEI CITY (TW); LI-PING YU, TAIPEI CITY (TW); HSIANG-CHING LEE, TAIPEI CITY (TW)

(21) Appl. No.: 16/110,186

(22) Filed: Aug. 23, 2018

(30) Foreign Application Priority Data
Jul. 9, 2018 (CN) 201810776438.7

(51) Int. Cl. A61B 3/10; A61B 5/00; A61B 5/01

(52) U.S. Cl. CPC A61B 3/10; A61B 5/01; A61B 5/0022

(57) ABSTRACT

A biomedical information processing system using an application program (APP) collaborated with an ocular temperature measurement technique includes an electronic device and a remote device. At least one user respectively holds an ocular temperature sensing device connected with the electronic device installed with an application program (APP). The electronic device exchanges information with the remote device. The ocular temperature sensing device acquires an instant ocular temperature measurement of a user, which is transmitted to the remote device through the APP. The remote device performs a characteristics comparison procedure according to a piece of comparison item information selected by the user on the APP to generate a comparison result. When the electronic device receives the comparison result from the remote device, the user can view the comparison result and create a tracking record through the electronic device for users to be aware of their eye health conditions at any time.

### BACKGROUND OF THE INVENTION

### 1. Field of the Invention

The present invention relates to a biomedical information processing system and a method for the same and, more particularly, to a biomedical information processing system using an application program collaborated with an ocular temperature measurement technique and a method for the same.

### 2. Description of the Related Art

Technological diversification brings forth social prosperity and advancement on the one hand and in the meantime also results in the issue of using 3C products too much. Recently, smart phones, tablet personal computers (PC), notebook computers and the like become widespread and seem to be all around us. However, certain eye conditions or even eye diseases, such as dry eye syndrome or nearsightedness, may arise from overuse of the 3C products over a long period of time. On the other hand, some medical experiments show that ocular surface temperature can reflect not only health conditions or pathological changes of eyes but other symptoms rather than dry eye syndrome or nearsightedness when ocular surface temperature is detected to be rising. Presently, before finding out the causes of eye conditions and diseases and getting medical treatment from professional medical institution, patients may have suffered from eye discomfort for a while. Such treatment delay certainly causes pain or inconvenience to busy modernized people in their daily life.

As disclosed in Taiwanese Patent No. 1597049, entitled “Wearable ocular surface temperature monitor apparatus and system using the same” (hereinafter called conventional technique), the apparatus is composed of a frame and an ocular temperature monitor module. The ocular temperature monitor module has a first temperature sensor and a temperature signal processing circuit. The first temperature sensor in installed inside the frame to measure an ocular surface temperature of a user's eye, provides an electrical signal to the temperature signal processing circuit, and transmits a wireless signal out for a mobile device to receive the wireless signal, such that the user's ocular temperature can be determined if it is abnormal and the user's ocular temperature can be instantly displayed.

As can be seen from the conventional technique, a wearable ocular surface temperature monitor apparatus, which takes the form of a spectacle and mainly detects one or more users' ocular surface temperatures, is provided. With the aid of the mobile device, users' ocular temperature can be determined if it is normal through a simple approach. However, the conventional technique can only allow users to be aware of their ocular surface temperature. If interested in digging deeper into the cause of disease or syndrome, users may take the measure of speculating the cause themselves or finding relevant information on the web instead of an actual visit to medical institution for diagnosis in person. The way of finding the cause of disease of syndrome is simply inconvenient, time-consuming and not timely enough to meet the need of the fast-paced contemporary people.

### SUMMARY OF THE INVENTION

An objective of the present invention is to provide a biomedical information processing system using an application program collaborated with an ocular temperature measurement technique and a method for the same, which employ the ocular temperature measurement technique and a cloud processing technique to perform a trending analysis targeting at ocular temperature variation of testees, compare the ocular temperature measurements with cloud information for users to efficiently acquire the analysis report for health conditions relevant to or observed from the eyes, such that eye symptoms or other eye-related diseases can be identified and prevented for fulfillment of preventive medicine.

To achieve the foregoing objective, the biomedical information processing method using an application program collaborated with an ocular temperature measurement technique, wherein the method is performed by an application program (APP) installed in an electronic device connected to a remote device and the method comprises:

selectively acquiring an instant ocular temperature measurement;

selectively uploading the instant ocular temperature measurement to the remote device;

transmitting a comparison command including a piece of comparison item information optionally having at least one characteristic item selected by a user of the electronic device to the remote device;

instructing the remote device to perform a characteristic comparison procedure to generate a comparison result according to the piece of comparison item information; and

receiving the comparison result and relevant information transmitted from the remote device.

Given the foregoing method, the remote device can store multiple pieces of testee information, and users just need to activate the APP in the electronic device connected to the remote device and select the piece of comparison item information from the APP. When the electronic device selectively acquires an instant ocular temperature measurement of a present user and uploads the measurement to the remote device, the remote device performs the characteristics comparison procedure to generate a comparison result according to the piece of comparison item information selected by the present user, and transmits the comparison result back to the electronic device. By virtue of the APP, the comparison result and relevant information can be checked at any time and a tracking record can be created. Attributable to the analysis of ocular temperature variation, the present invention can get hold of user's health conditions associated with or observed from the eyes.

To achieve the foregoing objective, the biomedical information processing system using an application program collaborated with an ocular temperature measurement technique includes an ocular temperature sensing device, an electronic device, and a remote device.

The ocular temperature sensing device selectively acquires an instant ocular temperature measurement of a present user and built in with a communication protocol.

The electronic device has an application program (APP) installed therein to connect to the ocular temperature sensing device through the communication protocol and receive the instant ocular temperature measurement.

The remote device stores multiple pieces of testee information and exchanges information with the electronic device through a network.

The APP in the electronic device allows the present user to configure basic information with multiple characteristic items of the present user and select a piece of comparison item information optionally including at least one characteristic item selected from the basic information on a default or runtime basis. When selectively acquiring the instant ocular temperature measurement, the electronic device selectively uploads the instant measurement to the remote device and transmits a comparison command including the piece of comparison item information to the remote device, the remote device then performs a characteristics comparison procedure to generate a comparison result according to the selectively acquired instant ocular temperature measurement and the comparison command, and transmits the comparison result and relevant information back to the electronic device for the present user to check the comparison result and the relevant information or create a tracking record at any time through the APP in the electronic device.

From the foregoing system architecture, an operator of the system can store all pieces of testee information at the remote device, users can launch the APP in the electronic device to connect with the ocular temperature sensing device through the communication protocol and selectively measure ocular temperature with the ocular temperature sensing device to acquire the instant ocular temperature measurement, select the piece of comparison item information through operation of the APP, and selectively upload the measurement and transmit the comparison command to the remote device, and the remote device performs the characteristics comparison procedure according to the measurement and the comparison command. When the electronic device receives the comparison result and the relevant information transmitted back from the remote device, users can check the comparison result or create a tracking record through the APP. Accordingly, users' awareness of eye health conditions at any time can be always there through the analysis of ocular temperature variation.

Other objectives, advantages and novel features of the invention will become more apparent from the following detailed description when taken in conjunction with the accompanying drawings.

### BRIEF DESCRIPTION OF THE DRAWINGS

FIG. 1 is a functional block diagram showing system architecture of a first embodiment of a biomedical information processing system in accordance with the present invention;

FIG. 2 is a functional block diagram showing system architecture of a second embodiment of a biomedical information processing system in accordance with the present invention;

FIG. 3 is a schematic diagram showing a state of logging in an application program installed in an electronic device of the biomedical information processing system in FIG. 1 or 2;

FIG. 4 is a schematic diagram showing a state of selecting a pairing device through the application in FIG. 3;

FIG. 5 is a schematic diagram showing a state of running a tutorial for operation of an ocular temperature sensing device from the application in FIG. 3;

FIG. 6 is a trending diagram showing a state of displaying an ocular temperature analysis result on the application in FIG. 3;

FIG. 7 is trending diagram showing a state of displaying another ocular temperature analysis result on the application in FIG. 3;

FIG. 8 is a schematic diagram showing a state of tracking records through the application in FIG. 3;

FIG. 9 is a flow diagram of a biomedical information processing method in accordance with the present invention; and

FIG. 10 is a flow diagram of a step of performing a characteristics comparison procedure in FIG. 9.

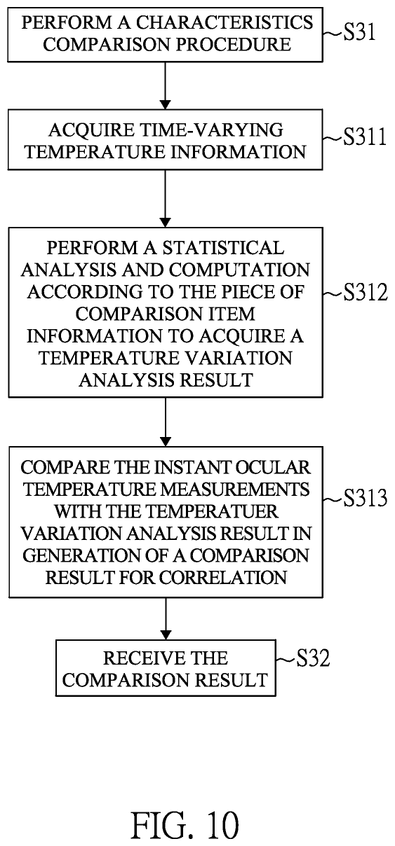

### DETAILED DESCRIPTION OF THE INVENTION

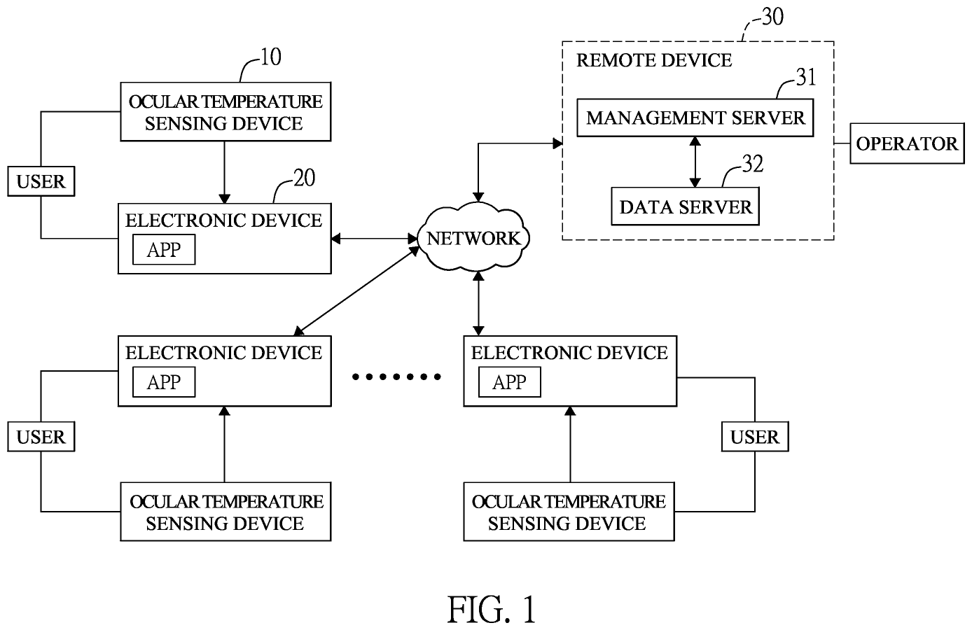

With reference to FIG. 1, a biomedical information processing system using an application program collaborated with an ocular temperature measurement technique in accordance with the present invention includes an ocular temperature sensing device 10 and an electronic device 20 for a user, and a remote device 30 for an operator. The remote device 30 stores multiple pieces of testee information and exchange information with the electronic device 20 through a network. In the present embodiment, the user holds the ocular temperature sensing device 10 and the electronic device 20 for operation, and the electronic device 20 is a handheld mobile device, a smart phone, a tablet PC or a notebook computer.

The ocular temperature sensing device 10 serves to acquire an instant ocular temperature measurement and is built in with a communication protocol. The electronic device 20 has a dedicated application program (hereinafter called APP) installed therein to pair with the ocular temperature sensing device 10 for connection through the communication protocol to receive the instant ocular temperature measurement. In the present embodiment, the communication protocol is a Bluetooth protocol, a WiFi (Wireless Fidelity) protocol, or a protocol for wired communication.

The APP in the electronic device 20 allows the user to configure basic information with multiple characteristic items of a user and select a piece of comparison item information optionally including at least one characteristic item selected from the basic information on a default or runtime basis. When selectively acquiring the instant ocular temperature measurement, the electronic device 20 selectively uploads the instant ocular temperature measurement to the remote device 30 and transmits a comparison command including the piece of comparison item information to the remote device 30. The remote device 30 then performs a characteristics comparison procedure to generate a comparison result according to the selectively acquired instant ocular temperature measurement and the comparison command, and transmits the comparison result and relevant information back to the electronic device 20 for the user to check the comparison result and the relevant information or create a tracking record at any time through the APP in the electronic device 20.

The remote device 30 for the operator includes a management server 31 and a data server 32. With further reference to FIG. 1, the management server 31 and the data server 32 are mutually connected. The management server 31 serves to store multiple pieces of testee information of multiple users and multiple characteristics corresponding to the respective characteristic items in the basic information of each user. The data server 32 stores multiple pieces of account information of the multiple users and the multiple ocular temperature measurements of the multiple users. When the electronic device 20 selectively acquires the instant ocular temperature measurement of a present user and selectively uploads the instant measurement to the data server 32 of the remote device 30 and transmits the comparison command including the piece of comparison item information to the management server 31 of the remote device 30, the management server 31 and the data server 32 of the remote device 30 perform the characteristics comparison procedure to generate the comparison result.

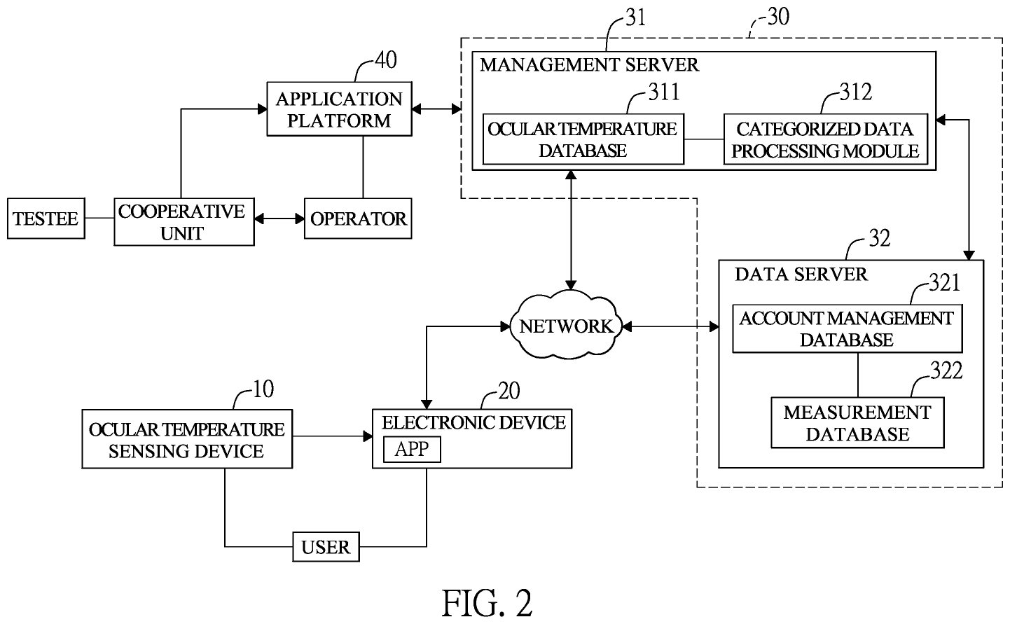

With reference to FIG. 2, the management server 31 includes an ocular temperature database 311 and a categorized data processing module 321. The ocular temperature database 311 and the categorized data processing module 321 are mutually connected. The data server 32 includes an account management database 321 and a measurement database 322. The account management database 321 and the measurement database 322 are mutually connected. The ocular temperature database 311 is built up with multiple pieces of screened comparison item information and multiple characteristics corresponding thereto. The categorized data processing module 312 serves to process the multiple pieces of screened comparison item information and the characteristics in the ocular temperature database 311. The account management database 321 serves to store the piece of account information of each user registered through the APP. The measurement database 322 serves to store the instant ocular temperature measurements associated with the account information of each registered user. In the present embodiment, the multiple pieces of screened comparison item information and the characteristics corresponding thereto can be downloaded from an application platform 40 provided by the operator. The application platform 40 may be a portal website for a cooperative unit, such as a hospital, a school, a vendor or the like, and the operator to log in. The cooperative unit may transmit the multiple pieces of testee information to the remote device 30 through the application platform 40 to update and expand the multiple pieces of screened comparison item information and the characteristics corresponding thereto in the ocular temperature database 311.

Hence, after the electronic device 20 selectively acquires the instant ocular temperature measurement of the present user, the APP of the electronic device 20 selectively uploads the instant measurement to the data server 32 of the remote device 30. The account management database 321 of the data server 32 stores the instant measurement, if available, in a corresponding position of the measurement database 322 according to the piece of account information of the present user. Meanwhile, the APP of the electronic device 20 transmits the comparison command to the management server 31 of the remote device 30 according to the piece of comparison item information configured by the present user for the categorized data processing module 312 of the management server 31 to acquire a corresponding piece of comparison item information and characteristics corresponding thereto in the ocular temperature database 311. Subsequently, a historical ocular temperature measurement associated with the present user in the data server 32 is transmitted to the management server 31. The categorized data processing module 312 performs the characteristics comparison procedure with the historical ocular temperature measurement, the corresponding piece of comparison item information and the corresponding characteristics. When acquiring the comparison result, the remote device 30 then transmits the comparison result back to the electronic device 20.

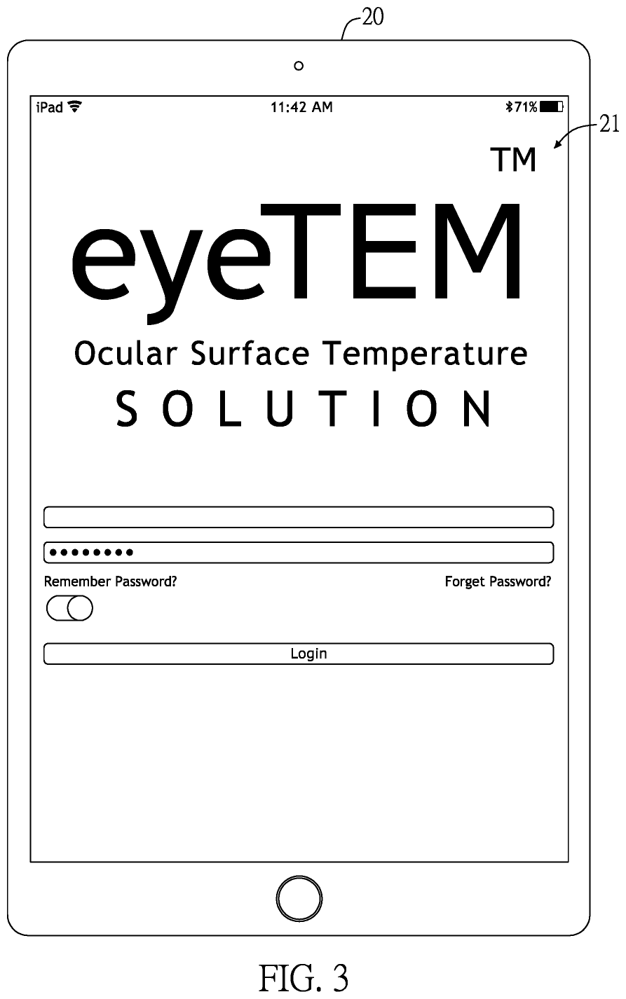

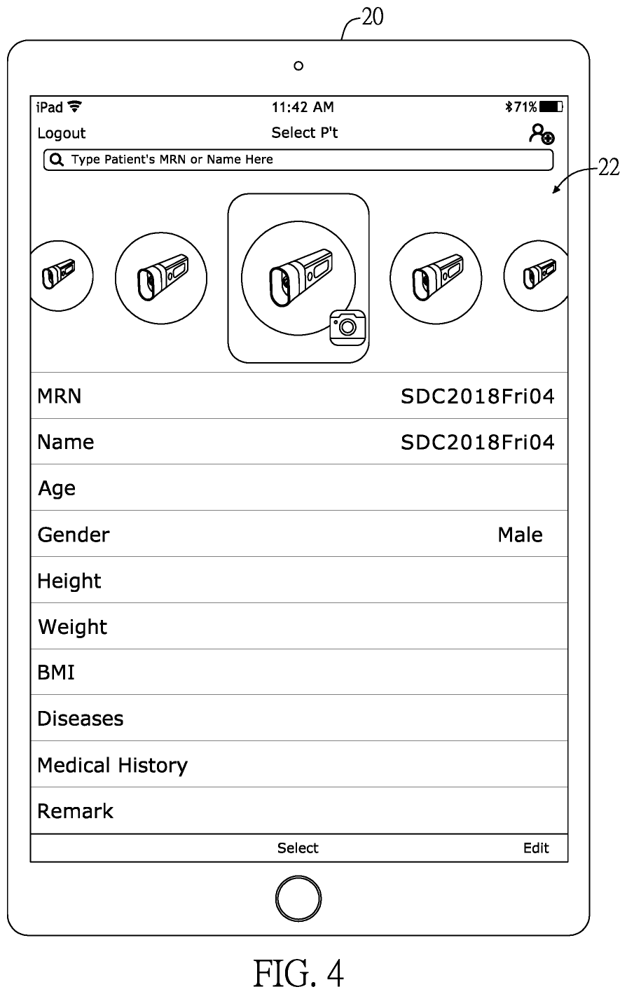

To elaborate operation of the biomedical information processing system, with reference to FIGS. 3 and 4, the present user can launch the APP on the electronic device 20 and activate a login module 21 of the APP for the user to input the piece of account information through the login module 21 and further activates a pairing and characteristics module 22. The present user can select one of multiple ocular temperature sensing devices to be paired through the pairing and characteristics module 22 for the electronic device 20 to pair with the selected ocular temperature sensing device 10 through the communication protocol. The pairing and characteristics module 22 further allows the present user to configure the basic information of the user and select the piece of comparison item information on the default or runtime basis. The piece of comparison item information may include age, gender, physical conditions, diseases, and the like.

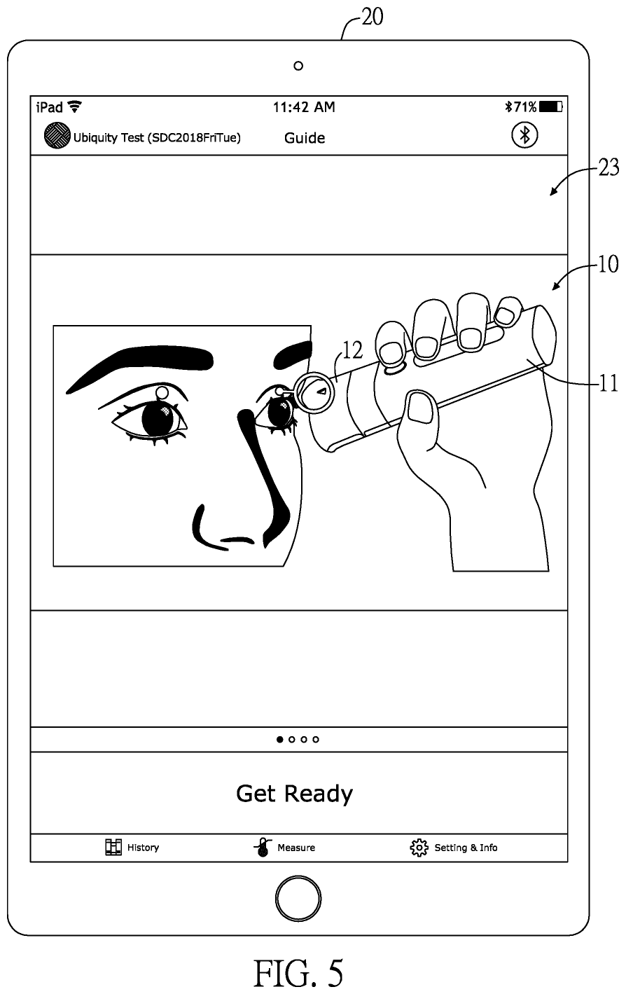

After configuration of the piece of comparison item information is completed, with reference to FIG. 5, the APP of the electronic device 20 further provides a tutorial 23 for operation of the ocular temperature sensing device 10 to facilitate user's comprehension in operating the ocular temperature sensing device 10. In the present embodiment, the ocular temperature sensing device 10 is a handheld device and has a grip portion 11 being cylindrical and an eyelid positioning member 12. The eyelid positioning member 12 is mounted on a top of the grip portion 11. During operation, the eyelid positioning member 12 of the ocular temperature sensing device 10 is directed to the user's eyelid to abut against an upper portion of the eyelid, such that the eyelid can be lifted up with the eye exposed such that the ocular temperature sensing device 10 can rapidly and conveniently measure the temperature at the center of the ocular surface of the eye.

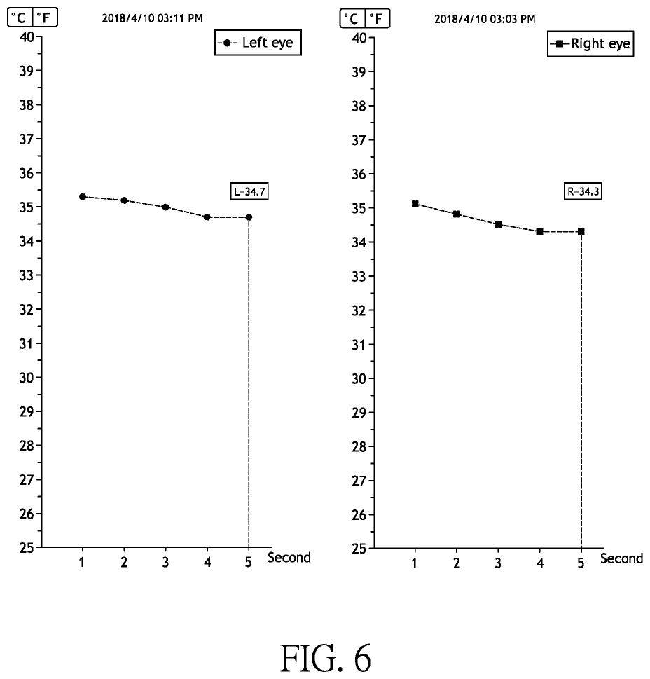

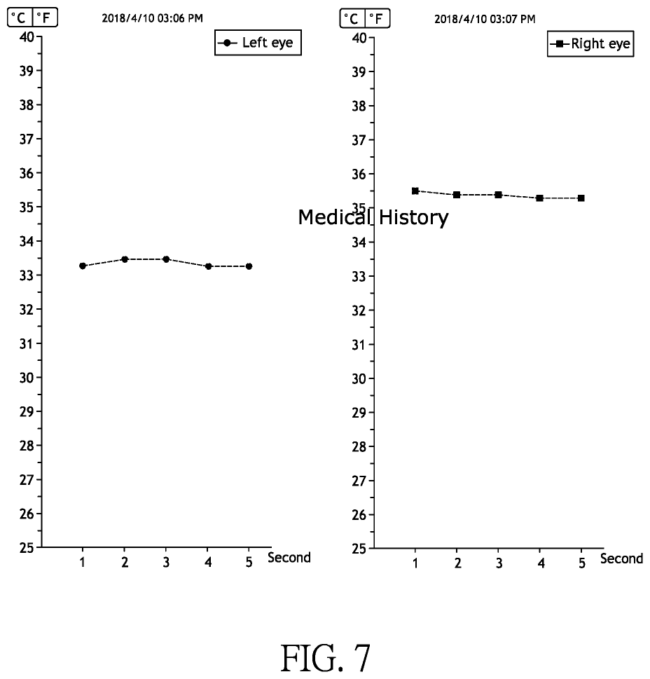

With reference to FIGS. 6 and 7, after the ocular temperature sensing device 10 transmits the multiple instant ocular temperature measurements measured over a period of time to the electronic device 20, the electronic device 20 records an average ocular temperature at each second over the period of time according to the instant ocular temperature measurement. The average ocular temperature at each second is obtained by taking an average of corresponding ocular temperatures measured within the second. The electronic device 20 then groups all the average ocular temperatures over the period of time as time-varying temperature information, and uploads the time-varying temperature information along with the piece of account information and the piece of comparison item information to the remote device 30. In the present embodiment, the time-varying temperature information is represented by a time-temperature curve with measured ocular temperatures varying over time.

When the APP of the electronic device 20 uploads the time-varying temperature information to the data server 32 of the remote device 30 and the comparison command including the piece of comparison item information configured by the user to the management server 31 of the remote device 30, the categorized data processing module 312 of the management server 31 retrieves corresponding piece of comparison item information and corresponding characteristics in the ocular temperature database 311 and the data server 32 transmits the historical ocular temperature measurement associated with the present user to the management server 31, such that the categorized data processing module 312 performs the characteristics comparison procedure with the historical ocular temperature measurement, the corresponding piece of comparison item information, and the corresponding characteristics. After a comparison result is obtained, the remote device 30 transmits the comparison result back to the electronic device 20. Additionally, the APP of the electronic device 20 can display the time-varying temperature information to show variation of ocular temperatures measured over the period of time and in the past, or obtain an ocular temperature variation rate, a highest ocular temperature, a lowest ocular temperature, a temperature range within which the ocular temperature measurements are situated.

Furthermore, the characteristics comparison procedure first acquires the time-varying temperature information from the instant ocular temperature measurement, then performs a statistical analysis and computation according to at least one characteristic (the characteristic may be purely gender or a certain disease) to acquire a temperature variation analysis result, compares the instant ocular temperature measurement of the present user with the temperature variation analysis result in generation a comparison result for correlation, and eventually transmits the comparison result for correlation to the electronic device 20. Given a female user over thirty as an example, the corresponding characteristics in the piece of comparison item information include age and gender. The age and the gender associated with the present user are provided for the statistical analysis and computation so as to obtain the temperature variation analysis result as a comparison with the instant ocular temperature measurement of the present user. In the present embodiment, the temperature variation analysis result includes a temperature range with all ocular temperatures measured therein associated with at least one characteristic, a temperature variation trend, and the like for the user to compare with.

To further explain how to acquire the time-varying temperature information, perform the statistical analysis and computation according to at least one characteristic, and generate a comparison result for correlation. With reference to FIGS. 6 and 7, given an instant ocular temperature measurement having multiple temperatures (e.g. 1,000 temperature values) measured over a sampling time period in a few seconds (e.g. 5 seconds) with respect to an identical characteristic as an example, the instant ocular temperature measurement is calculated to obtain the time-varying temperature information with an average ocular temperature at each second within the sampling time period, and a method including but not limited to a linear regression method is applied to the time-varying temperature information for computation in generation of a straight line. The slope of the straight line is a basis of comparison for the instant ocular temperature measurement associated with the characteristic. In the mean time, multiple error bars respectively drawn at multiple seconds of the sampling time period in the temperature variation analysis result can be mutually connected to define a correlation range within which the instant ocular temperature measurement should fall. Once a specific instant ocular temperature measurement associated with the characteristic falls within the correlation range, it indicates that the specific instant ocular temperature measurement has a higher degree of correlation with the temperature variation analysis result, or in other words, the specific instant ocular temperature measurement is close within a range to an average ocular temperature measurement measured from other users and calculated based on a statistical approach.

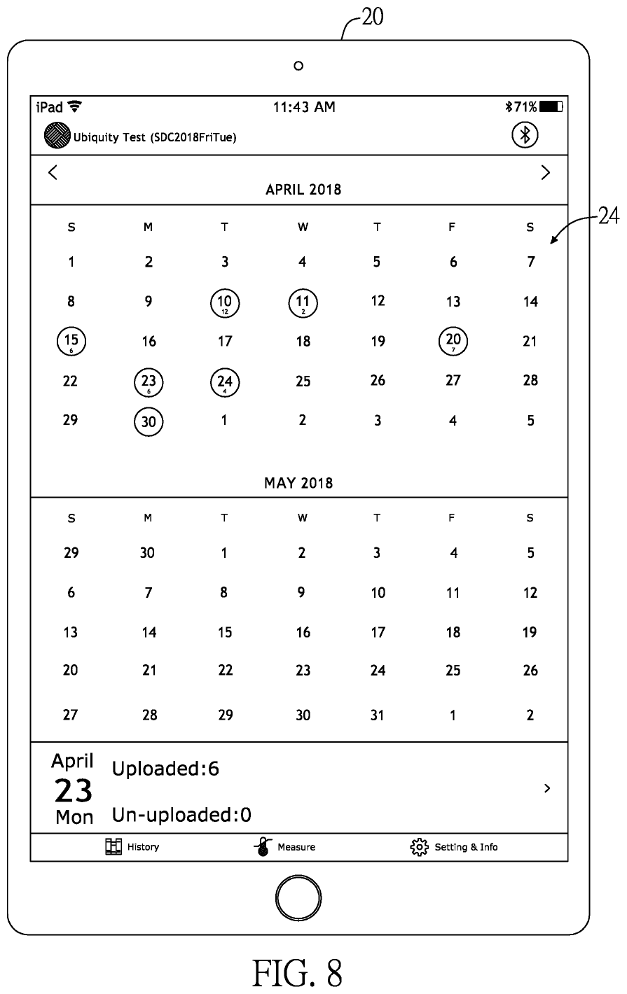

With reference to FIG. 8, the APP of the electronic device 20 further provides a date-based recording module 24 for the present user to click any date on the APP of the electronic device 20, such that the date-based recording module 24 can show entire instant ocular temperature measurements of the present user at the date for the present user to view. The present user can upload the entire instant ocular temperature measurements of the date from the APP of the electronic device 20 to the remote device 30 for backup or for other subsequent processing. The date-based recording module 24 has a usage count recording function to acquire a user's usage count which can be taken as a criterion applied to an incentive plan.

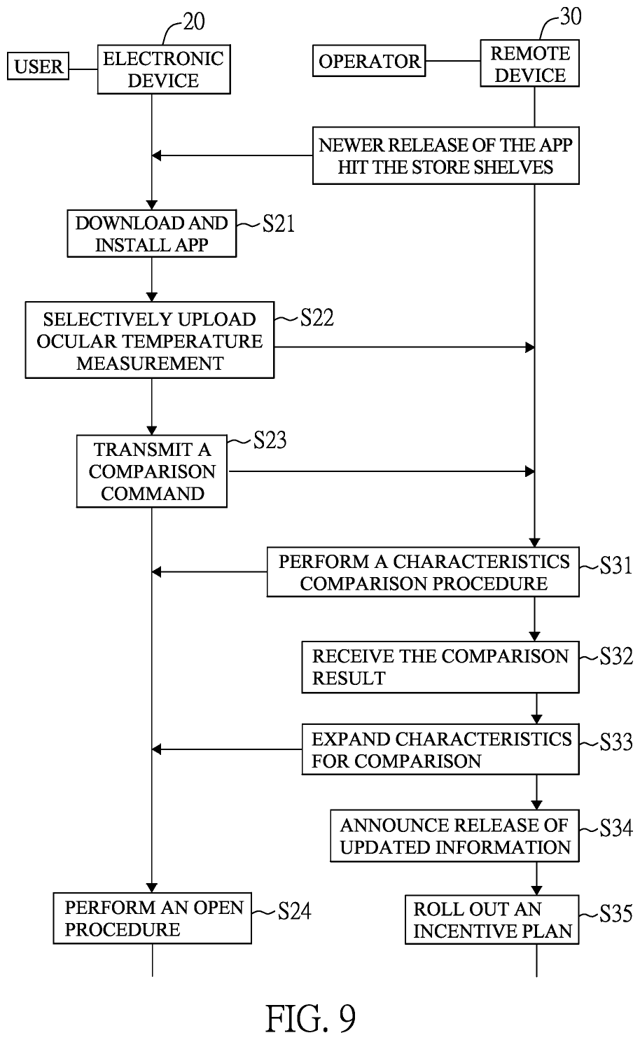

Based on the description of the foregoing embodiments, with reference to FIG. 9, a biomedical information processing method using an application program collaborated with an ocular temperature measurement technique in accordance with the present invention can be further derived. For implementation of the method, the electronic device 20 of the user is connected to the remote device 30 of an operator, and the operator can allow a newer release of the APP to hit the store shelves at any time for the user to download. The method is performed by the electronic device 20 and includes the following steps.

Step S21: Download and install the APP in the electronic device to selectively acquire an instant ocular temperature measurement.

Step S22: Selectively upload the instant ocular temperature measurement to the remote device 30.

Step S23: Transmit a comparison command including a piece of comparison item information optionally having at least one characteristic item selected by the user to the remote device 30.

Step S31: Instruct the remote device 30 to perform a characteristics comparison procedure to generate a comparison result according to the piece of comparison item information.

Step S32: Receive the comparison result and relevant information transmitted from the remote device.

The method further includes the following steps when the operator intends to expand the characteristics for comparison.

Step S33: Expand characteristics for comparison through the remote device 30.

Step S34: Announce release of updated information.

Step S35: Roll out an incentive plan.

The method further includes the following step when the electronic device 20 receives the updated information.

Step S24: Perform an open procedure to acquire expanded characteristics.

Step S31 further includes the following sub-steps.

Step S311: Acquire time-varying temperature information. The APP of the electronic device 20 records an average ocular temperature at each second over a period of time according to the instant ocular temperature measurement. The average ocular temperature at each second is obtained by taking an average of multiple ocular temperatures measured over the second. The electronic device 20 then groups all the average ocular temperatures over the period of time as the time-varying temperature information, and uploads the time-varying temperature information to the remote device 30

Step S312: Perform a statistical analysis and computation according to at least one characteristic to acquire a temperature variation analysis result through the remote device 30.

Step S313: Compare the instant ocular temperature measurements with the temperature variation analysis result in generation of a comparison result for correlation through the remote device 30.

In the present embodiment, the temperature variation analysis result includes a temperature range of ocular temperatures measured associated with a characteristic, a temperature variation trend, and the like.

By virtue of the foregoing method, the remote device 30 stores the multiple pieces of testee information, and the APP of the electronic device 20 of the user is connected to the remote device 30 for the user to select the piece of comparison item information. When the electronic device 20 selectively acquires the instant ocular temperature measurements of the user and selectively uploads the measurements to the remote device 30, the remote device 30 performs the characteristics comparison procedure according to the piece of comparison item information configured by the user in generation of the comparison result, and transmits the comparison result back to the electronic device 20. The comparison result and relevant information at any time can be checked and stored as records for tracking through the APP of the electronic device 20. In view of comparison and analysis of ocular temperature variation, users can instantly get hold of their eye health conditions.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A biomedical information processing method using an application program collaborated with an ocular temperature measurement technique, wherein the method is performed by an application program (APP) installed in an electronic device connected to a remote device and the method comprises:
selectively acquiring an instant ocular temperature measurement;
selectively uploading the instant ocular temperature measurement to the remote device;
transmitting a comparison command optionally including a piece of comparison item information having at least one characteristic item selected by a user of the electronic device to the remote device;
instructing the remote device to perform a characteristic comparison procedure to generate a comparison result according to the piece of comparison item information; and
receiving the comparison result and relevant information transmitted from the remote device.

2. The method as claimed in claim 1, wherein the step of instructing the remote device to perform a characteristic comparison procedure further comprises:
acquiring time-varying temperature information;
performing a statistical analysis and computation according to the piece of comparison item information to acquire a temperature variation analysis result; and
comparing the instant ocular temperature measurement with the temperature variation analysis result in generation of a comparison result for correlation.

3. The method as claimed in claim 2, wherein in the step of acquiring the time-varying temperature information, the APP of the electronic device records an average ocular temperature at each second over a period of time according to the instant ocular temperature measurement, the average ocular temperature at each second is obtained by taking an average of multiple ocular temperatures measured over the second, and the electronic device then groups all the average ocular temperatures over the period of time as the time-varying temperature information, and uploads the time-varying temperature information to the remote device.

4. The method as claimed in claim 2, wherein in the step of comparing the instant ocular temperature measurement with the temperature variation analysis result in generation of a comparison result for correlation, multiple error bars respectively drawn at multiple seconds of a sampling time period in the temperature variation analysis result are mutually connected to define a correlation range within which the instant ocular temperature measurement falls, and when the instant ocular temperature measurement associated with the at least one characteristic falls within the correlation range, it indicates that the instant ocular temperature measurement has a higher degree of correlation with the temperature variation analysis result.

5. The method as claimed in claim 2, wherein the temperature variation analysis result includes a temperature range with ocular temperatures measured therein associated with one of the optionally selected at least one characteristic item of the piece of comparison item information or a temperature variation trend.

6. The method as claimed in claim 3, wherein the temperature variation analysis result includes a temperature range with ocular temperatures measured therein associated with one of the optionally selected at least one characteristic item of the piece of comparison item information or a temperature variation trend.

7. The method as claimed in claim 4, wherein the temperature variation analysis result includes a temperature range with ocular temperatures measured therein associated with one of the optionally selected at least one characteristic item of the piece of comparison item information or a temperature variation trend.

8. A biomedical information processing system using an application program collaborated with an ocular temperature measurement technique, comprising:
an ocular temperature sensing device selectively acquiring an instant ocular temperature measurement of a present user and built in with a communication protocol;
an electronic device having an application program (APP) installed therein to connect to the ocular temperature sensing device through the communication protocol and receive the instant ocular temperature measurement; and
a remote device storing multiple pieces of testee information and exchanging information with the electronic device through a network;
wherein the APP in the electronic device allows the present user to configure basic information with multiple characteristic items of the present user and select a piece of comparison item information optionally including at least one characteristic item selected from the basic information on a default or runtime basis, when selectively acquiring the instant ocular temperature measurement, the electronic device selectively uploads the instant measurement to the remote device and transmits a comparison command including the piece of comparison item information to the remote device, the remote device then performs a characteristics comparison procedure to generate a comparison result according to the selectively acquired instant ocular temperature measurement and the comparison command, and transmits the comparison result and relevant information back to the electronic device for the present user to check the comparison result and the relevant information or create a tracking record at any time through the APP in the electronic device.

9. The system as claimed in claim 8, wherein the remote device includes:
a management server storing multiple pieces of testee information of the multiple users and multiple characteristics corresponding to the respective characteristic items in the basic information of each user; and
a data server connected to the management server, and storing multiple pieces of account information of the multiple users and the instant ocular temperature measurements of the multiple users;
wherein when the electronic device selectively acquires the instant ocular temperature measurement from the present user, selectively uploads the instant ocular temperature measurement to the data server, and transmits the comparison command including the piece comparison item information to the management server, the management server and the data server perform the characteristics comparison procedure to generate the comparison result.

10. The system as claimed in claim 9, wherein
the management server includes:
an ocular temperature database built in with multiple pieces of screened comparison item information and characteristics corresponding thereto; and
a categorized data processing module connected to the ocular temperature database and processing a corresponding piece of screened comparison item information and the characteristics corresponding thereto in the ocular temperature database; and
the data server includes:
an account management database storing the piece of account information of each user registered through the APP; and
a measurement database connected to the account management database storing the instant ocular temperature measurement associated with account information of each registered user.

11. The system as claimed in claim 10, wherein the APP of the electronic device includes:
a login module allowing each user to input a corresponding piece of account information therethrough;
a pairing and characteristics module allowing the present user to configure the basic information of each user, select the piece of comparison item information, and select one of multiple ocular temperature sensing devices to be paired therethrough for the electronic device to pair with the selected ocular temperature sensing device;
a tutorial for operation of the ocular temperature sensing device; and
a date-based recording module showing entire instant ocular temperature measurements of the present user at a selected date for the present user to view.

12. The system as claimed in claim 8, wherein the ocular temperature sensing device is a handheld device and has:
a grip portion being cylindrical; and
an eyelid positioning member mounted on a top of the grip portion.

13. The system as claimed in claim 9, wherein the ocular temperature sensing device is a handheld device and has:
a grip portion being cylindrical; and
an eyelid positioning member mounted on a top of the grip portion.

14. The system as claimed in claim 10, wherein the ocular temperature sensing device is a handheld device and has:
a grip portion being cylindrical; and
an eyelid positioning member mounted on a top of the grip portion.

15. The system as claimed in claim 11, wherein the ocular temperature sensing device is a handheld device and has:
a grip portion being cylindrical; and
an eyelid positioning member mounted on a top of the grip portion.
